(19) United States
(12) Patent Application Publication
(10) Pub. No.: US 2024/0376532 A1
(43) Pub. Date: Nov. 14, 2024

(54) APPARATUSES, SYSTEMS, AND METHODS FOR DETECTING BIOMARKERS ASSOCIATED WITH RISK OF PRESSURE INJURIES

(71) Applicants: THE UNITED STATES GOVERNMENT AS REPRESENTED BY THE DEPARTMENT OF VETERANS AFFAIRS, Washington, DC (US); CASE WESTERN RESERVE UNIVERSITY, Cleveland, OH (US)

(72) Inventor: Katherine M. Bogie, Shaker Heights, OH (US)

(21) Appl. No.: 18/691,868

(22) PCT Filed: Sep. 14, 2022
(86) PCT No.: PCT/US2022/043469

§ 371 Date: Mar. 13, 2024

Related U.S. Application Data
(60) Provisional application No. 63/243,992, filed on Sep. 14, 2021.

(51) Int. Cl. C12Q 1/6844; C12Q 1/6876

(52) U.S. Cl. CPC C12Q 1/6844; C12Q 1/6876; C12Q 2600/158

(57) ABSTRACT

Risk of pressure injuries can be assessed by detecting, in a biological sample, a respective level of at least one biomarker associated with pressure injuries. The biological sample can be, for example, whole blood or other bodily fluids. The at least one biomarker can comprise fatty acid binding protein-3 (FABP3) and/or fatty acid binding protein-4 (FABP4). The biomarkers can be detected by reverse transcription loop-mediated isothermal amplification (RT-LAMP) methods. The biomarkers can be detected by a microfluidics-based biochip.

### CROSS-REFERENCE TO RELATED APPLICATIONS

This application claims priority to, and the benefit of the filing date of, U.S. Provisional Patent Application No. 63/243,992, filed Sep. 14, 2021, the entirety of which is hereby incorporated by reference herein.

### FIELD

This disclosure relates to systems and methods for detecting risk of pressure injuries.

### BACKGROUND

Pressure injuries (PrIs) remain one of the most significant and frequent secondary complications for active duty military and Veterans with spinal cord injury (SCI). Development of a PrI has a devastating impact on quality of life, often leading to prolonged hospitalization, further complications and even death. PrI development causes pain, discomfort, negative body image etc., leading to greater isolation and poor quality of life (QoL). It was found that community-acquired PrI are among the most common causes of rehospitalization. The cost burden of PrI is significant. Over 5 years ago the direct costs of Stage 4 PrI exceeded $100,000 per PrI. The most recent estimates for chronic wound care indicate that annual costs range between $6 billion and $15 billion, and may be as high as 5% of all healthcare expenditures. There are also indirect costs due to loss of income, productivity, progress towards rehabilitation and vocational goals, independence, self-esteem, and sense of self-worth. It has been estimated that PrI prevention is approximately 2.5 times more economical than treatment.

The treatment costs of PrI particularly impact the Veterans Heath Administration (VHA) because they provide a lifetime of care for Veterans with SCI for at least 20% of the 300,000 persons with SCI in the USA. The Office of Inspector General (OIG) completed an evaluation of PrI prevention and management at VHA facilities in 2015. The OIG report highlights recommendations for innovative PrI risk assessment, and care plan options to improve quality of care provided to Veterans. A recent Congressional report also highlighted concern regarding hospitalization costs for treatment of Veterans with PrI.

The development of a PrI may occur rapidly but takes many weeks or months to heal. Many individuals with SCI are affected by tissue breakdown and recurrent PrI development following injury. A continuous cycle of recurring PrIs often requires extended bedrest and has a devastating impact on overall health and quality of life (QoL). There are multiple benefits, from personal to societal, to reliably identifying the highest risk individuals, so that interventions can be effectively utilized and the insidious cycle of PrI recurrence minimized.

### SUMMARY

Risk of pressure injuries can be assessed by detecting, in a biological sample, a respective level of at least one biomarker associated with pressure injuries. The biological sample can be, for example, blood, saliva or other bodily fluids. The at least one biomarker can comprise fatty acid binding protein-3 (FABP3) and/or fatty acid binding protein-4 (FABP4) and/or other markers of interest. The biomarkers can be detected by reverse transcription loop-mediated isothermal amplification (RT-LAMP) methods. The biomarkers can be detected by a microfluidics-based biochip.

Apparatuses for performing such methods are also disclosed.

### BRIEF DESCRIPTION OF THE DRAWINGS

FIG. TA shows an apparatus for detecting biomarkers as disclosed herein with an exploded view of said apparatus. FIG. 1B shows a top view of said apparatus with a penny for size reference.

FIG. 2 shows a schematic diagram of system for using the apparatus of FIG. 1 and a data plot illustrating readouts of the system.

FIG. 3 shows an experimental design of RT-LAMP assay for detection of PrI risk circulatory biomarkers.

FIG. 4 shows gluteal muscle area percentage intramuscular adipose tissue content for the example. Error bars show standard deviation.

FIG. 5A shows 3D reconstruction gluteal muscle composition at 9 years post injury: posterior views showing increase in intramuscular fat over one year for a 58 year old male with chronic complete SCI and a history of severe tissue breakdown. Gluteal muscle bilaterally 38% IMAT. FIG. 5B shows a 3D reconstruction of the 58-year old's gluteal muscle composition at 10 years post injury

FIGS. 6A-6D shows calibrated relative normalized quantities (CNRQ) of fatty acid binding proteins and inflammatory biomarkers of interest in gluteal muscle and whole blood samples between a group with no PrI history and a group with recurrent PrI history. FIG. 6A shows fatty acid binding proteins in gluteal muscle; FIG. 6B shows inflammatory biomarkers in gluteal muscle; FIG. 6C shows fatty acid binding proteins in whole blood; FIG. 6D shows inflammatory biomarkers in whole blood samples; and FIG. 6E shows the legend for FIGS. 6A-6D.

FIG. 7 is a block diagram of an exemplary operating environment showing a computing device in accordance with embodiments disclosed herein.

FIG. 8 is a block diagram of an exemplary apparatus for detecting biomarkers as disclosed herein.

### DETAILED DESCRIPTION

The disclosed system and method may be understood more readily by reference to the following detailed description of particular embodiments and the examples included therein and to the Figures and their previous and following description.

It is to be understood that the terminology used herein is for the purpose of describing particular embodiments only and is not intended to limit the scope of the present invention which will be limited only by the appended claims.

It must be noted that as used herein and in the appended claims, the singular forms “a,” “an,” and “the” include plural references unless the context clearly dictates otherwise. Thus, for example, reference to “a biomarker” includes one or more of such biomarkers, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Optionally, in some aspects, when values are approximated by use of the antecedents “about,” “substantially,” or “generally,” it is contemplated that values within up to 15%, up to 10%, or up to 5% (above or below) of the particularly stated value or characteristic can be included within the scope of those aspects.

Unless defined otherwise, all technical and scientific terms used herein have the same meanings as commonly understood by one of skill in the art to which the disclosed apparatus, system, and method belong. Although any apparatus, systems, and methods and materials similar or equivalent to those described herein can be used in the practice or testing of the present apparatus, system, and method, the particularly useful methods, devices, systems, and materials are as described.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### INTRODUCTION

Pressure injuries (PrI) are localized damage to the skin and/or underlying tissue, as a result of pressure or pressure in combination with shear. Pressure injuries usually overlie a bony prominence or are related to a medical or other device. Prolonged exposure to pressure with or without shear can cause capillary occlusion, tissue necrosis, and eventually death. Pressure injuries can typically be categorized into 4 stages. The 4 stages are generally categorized as follows: In Stage 1, a red, blue, or purplish area first appears on the skin like a bruise. In Stage 2, the bruise becomes an open sore that looks like an abrasion or blister. The skin around the wound can be discolored and the area is painful. In Stage 3, the sore deepens and looks like a crater, often with dark patches of skin around the edges. In Stage 4, the damage extends to the muscle, bone, or joints and can cause a serious infection of the bone, known as osteomyelitis. Stage 4 can also lead to sepsis. In various aspects of the present disclosure, pressure injury can be detected at or before stage 1.

Tissue breakdown and PrI development following spinal cord injury (SCI) can be prevented by maintaining healthy viable tissue under applied loads. Interface pressures need to be kept low, and duration of loading needs to be kept short, to prevent tissue breakdown in the individual with reduced mobility. Specialized support surfaces and a regular pressure relief regimen can be used for PrI prevention. Adequate tissue resilience can be advantageous for viable tissue to be maintained under applied loads. For the full-time wheelchair user, maintaining adequate tissue resilience includes maintaining pelvic region tissue health while seated. Traditional techniques to mitigate PrI risk focus on reducing applied pressures and decreasing the duration of loading. Pressure relief can maintain adequate skin and skeletal muscle perfusion by recovery of blood flow through the local microvasculature. Pressure relief regimens can be determined based on an individual's risk characteristics. Some individuals with SCI exhibit an impaired response to repeated loading, characterized by slow and incomplete tissue recovery between load cycles and considerable variation of the applied pressure required to lower skin blood flow below a critical threshold. It remains unclear how frequently an individual with SCI needs to weight shift in order to avoid tissue damage. No universally effective pressure relief regimen has been identified. Pressure relief every 20 minutes is recommended but unfeasible for most individuals and rarely maintained rigorously. Continued high PrI incidence indicates that these standardized regimes are failing for many individuals at risk.

Many other factors related to PrI development present challenges, including, in particular, development of deep tissue PrI (DTPrI). The use of standard quantitative tissue health assessment techniques, such as interface pressure mapping, has limited relationship with risk for DTPrI because damage is not initiated at the surface. Modeling studies increase understanding of the stresses and strain that can cause DTPrI, but do not present clear expectations in a clinical setting. The reported incidence of DTPrI appears to be rising, which can be a function of clinician awareness. For the SCI population specifically, incidence and recurrence are often not directly correlated with routinely considered clinical factors such as injury level.

In addition, clinicians observe that some individuals appear to remain PrI free following injury, whereas others suffer from a continuous cycle of recurring PrIs. That is, within the increased risk population of all individuals with SCI, a portion of these individuals are at increased propensity for PrI development.

Methods disclosed herein have an associated order in which steps are described. However, the order should be understood to be exemplary rather than limiting, and other orders of operation should be understood to be included within this disclosure.

Disclosed herein are systems, apparatuses, and methods for detecting biomarkers associated with pressure injuries.

A method can comprise detecting, in a biological sample, a respective level of at least one biomarker associated with pressure injuries. The biological sample can be, for example, blood, saliva, or other bodily fluids, such as, for example and without limitation, urine. The at least one biomarker can comprise fatty acid binding protein-3 (FABP3) and/or fatty acid binding protein-4 (FABP4). In further or alternative aspects, the at least one biomarker can comprise Kruppel-like factor 4, resistin, cyclin D1, sirtuin 2, dysferlin 2B, pyruvate, dehydrogenase kinase-4, dystrophin, or adiponectin.

The respective level of the at least one biomarker associated with pressure injuries can be detected using reverse transcription loop-mediated isothermal amplification (RT-LAMP) methods. The RT-LAMP method can be performed on a microfluidics-based biochip to detect the respective level of the at least one biomarker associated with pressure injuries.

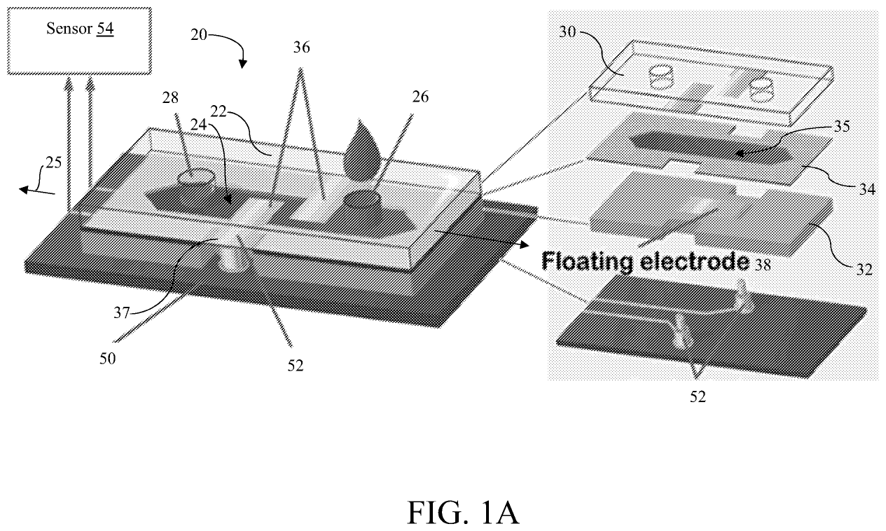

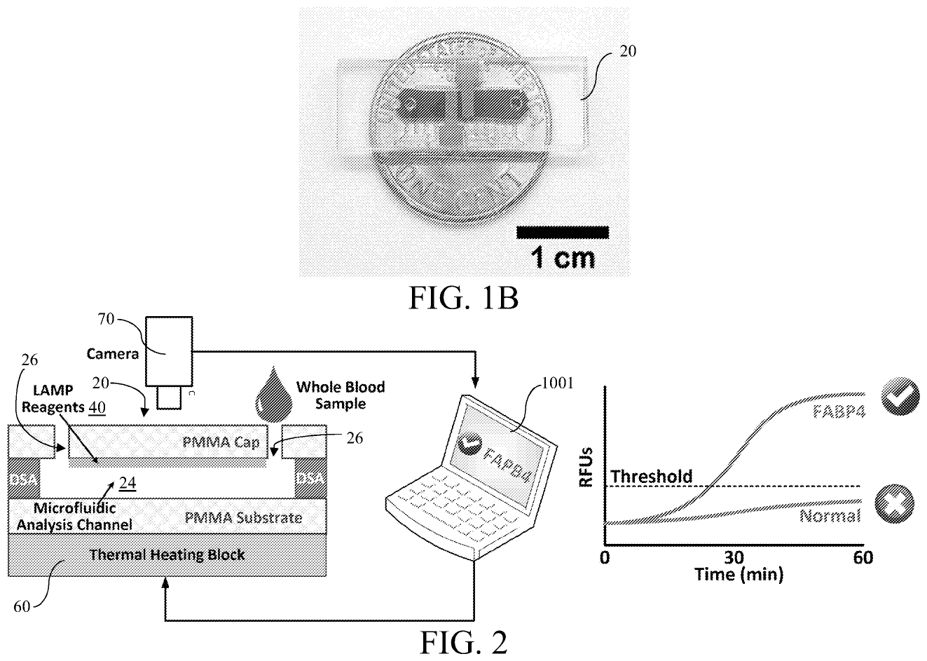

Referring to FIGS. 1A-2, in some aspects, an apparatus 10 can comprise a microfluidics-based biochip 20 that is configured to detect a respective level of at least one biomarker associated with pressure injuries in a biological sample, such as a blood sample, a saliva sample, and/or a urine sample. For example, the microfluidics-based biochip can be configured to detect a respective level of one or both of FABP3 or FABP4.

In some aspects, and with reference to FIGS. 1A, 2, and 8, the microfluidics-based biochip 20 can comprise a body 22 that defines a channel 24. In some aspects, the channel 24 can be elongate along a longitudinal axis 25. The body 22 can define an inlet 26 that is configured to receive a fluid sample (e.g., blood) and an outlet 28 that permits gas (e.g., air) to escape the channel so that the channel can receive the sample.

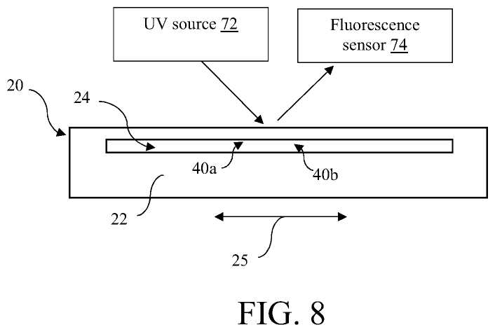

Optionally, the body 22 of the microfluidics-based biochip 20 can comprise a plurality of layers. For example, the body 22 can comprise a first layer 30, a second layer 32, and an intermediate layer 34 that is positioned between the first and second layers 30, 32. The intermediate layer 34 can define a cutout 35 that at least partly defines an outer periphery of the channel 24. In exemplary aspects, the first layer 30 can define the inlet 26 and the outlet 28, which can overlie the cutout 35 of the intermediate layer 34 (and thus, the channel 24). Optionally, the first and second layers 30, 32 can comprise polymer (e.g., poly(methyl methacrylate) (PMMA)). In some optional aspects, the intermediate layer 34 can comprise a double-sided adhesive (DSA) so that the intermediate layer 34 couples the first and second layers together. The first, second, and intermediate layers 30, 32, 34 can be coupled together to form the microfluidics-based biochip 20. In some aspects, the intermediate layer 34 can have a thickness of less than 1 mm (e.g., about 250 μm). The apparatus 10 can further comprise at least one reagent 40 (optionally, a plurality of reagents) within the channel 24. For example, the at least one reagent 40 can be disposed against a surface of the second layer 32 that at least partly defines the channel 24. For example, an upper surface of the second layer 32 can cooperate with the cutout 35 of the intermediate layer 34 to define the channel 24, with the upper surface of the second layer 32 defining a lower surface of the channel and the edges of the intermediate layer 34 that define the cutout 35 serving as walls of the channel. In some aspects, the reagents can comprise primers for performing RT-LAMP to detect a biomarker. In some aspects, the reagents can comprise primers for detecting a plurality of biomarkers. In some aspects, a plurality of different reagents can be spaced along the longitudinal axis 25 so that different locations along the longitudinal axis 25 can provide indications of different biomarkers. For example, referring also to FIG. 8, a first location can comprise a first set of reagents 40a (e.g., primers for performing RT-LAMP to detect a biomarker indicative of PrI), and a second location can comprise a second set of reagents 40b (e.g., primers for performing RT-LAMP to detect a reference gene).

Optionally, the apparatus 10 can comprise a plurality of electrodes that permit sensing electrical properties of a sample. For example, in some optional aspects, pH can be detected by the electrodes. In some aspects, the first layer 30 can comprise a plurality of sensing electrodes (optionally, a pair of sensing electrodes 36 that are spaced along, and in fluid communication with, the channel 24 (e.g., along the longitudinal axis 25 of the channel). The sensing electrodes 36 can be spaced by from about 0.2 mm to about 1 mm (e.g., about 0.4 mm). The sensing electrodes 36 can optionally have dimensions of about 0.6 mm by 0.6 mm. Optionally, first and second sensing electrodes can be coupled to opposing sides of the first layer 30 (e.g., on opposite sides of the channel 24). In further optional aspects, it is contemplated that the first and second sensing electrodes can be coupled or secured to a bottom surface of the first layer 30 (facing the channel 24). The second layer 32 can comprise a floating electrode 38 that extends along the channel 24. In some aspects, the floating electrode 38 can extend along the longitudinal axis 25 of the channel between the sensing electrodes 36, which can optionally be oriented perpendicularly or substantially perpendicularly to the longitudinal axis of the channel. When the sample is received with the channel, the pair of sensing electrodes 36 can be in electrical communication with each other and the floating electrode 38. That is, the floating electrode 38 and the sensing electrodes 26 can be on opposite sides of the sample fluid and can be in electrical communication through the fluid. As an example, the floating electrode can be formed with a thickness of 1000 angstroms and dimensions of 1.2 mm by 2.8 mm aligned with the sensing electrodes 36. Optionally, it is contemplated that the first layer 30 can be sufficiently transparent or translucent to permit observation of the sample within the channel 24.

With reference to FIG. 1A, the apparatus 10 can comprise a substrate 50 (e.g., a printed circuit board). The substrate can comprise contact pins 52 that are configured to electrically couple to the pair of sensing electrodes 36 via contact pads 37 of the sensing electrodes when the microfluidics-based biochip 20 is received thereon. In these aspects, it is contemplated that the second layer 32 and the intermediate layer 34 can define respective aligned slots or openings that receive a portion of a respective contact pin 52 and permit electrical coupling between the contact pin and a corresponding sensing electrode 36. The contact pins 52 can be in electrical communication with a sensor 54. In some optional aspects, the sensor 54 can be a pH sensor. In alternative aspects, the apparatus does not comprise electrodes for permitting sensing electrical properties of the sample, and the pair of sensing electrodes 36, floating electrode 38, contact pads, 37, and contact pins 52 can be omitted.

In some aspects, the sensor 54 can comprise an optical sensor. For example, in some optional aspects, the sensor 54 can be a colorimetric sensor. In some optional aspects, the sensor 54 can be a fluorescence sensor that is configured to detect an intensity of fluorescence.

In some aspects, the optical sensor can comprise a camera 70. The camera 70 can be in communication with a computing device 1001. Images captured by the camera 70 can be used to quantify colorimetric changes in the sample and a threshold that indicates the presence of the biomarker can be determined. In further or alternative aspects, an ultraviolet (UV) excitation source 72 (FIG. 8) positioned proximate to the microfluidics-based biochip 20 can be configured to cause fluorescence. A camera 70 or other optical sensor (e.g., a fluorescence sensor 74) can be configured to detect fluorescence. In this way, the biomarker can be optically detected. Optionally, the camera or other optical sensor can be configured to detect an intensity of the fluorescence.

As can be understood, conventionally, RT-LAMP has been used for detecting the presence or absence of a protein (e.g., SARS-COV-2). In contrast, the disclosed methods allow for determining a level of biomarker present in a given sample. More particularly, in some aspects, the sensor 54 can be configured to detect a level of biomarker(s) present in the sample, rather than just a presence or absence of the biomarker in the sample. For example, the intensity of the fluorescence captured by the optical sensor can be correlated to a particular level of biomarker present in the sample. As another example, a color, or brightness of the color, as captured by the optical sensor (e.g., camera) can be correlated to a particular level of biomarker present in the sample. Optionally, reagents can be provided that react with different colors (e.g., green and purple) to determine presence or absence of biomarkers as well as levels of biomarkers present. As further disclosed herein, the levels of biomarkers can be determined based on a database including information associating various historical measured/sensed outputs with corresponding levels of biomarkers that were present in those historical patients.

In some aspects, the apparatus 10 can comprise a heater 60 (e.g., a thermal heating block). A controller (e.g., a computing device 1001) can be in communication with the heater and can be configured to regulate the heater 60 to maintain the microfluidics-based biochip 20 at a particular temperature. For example, the apparatus 10 can be configured to maintain the microfluidics-based biochip 20 at a first temperature (e.g., from about 40° C. to about 90° C., or from about 60° C. to about 75° C., or about 65° C., or about 70° C.). After a predetermined time (e.g., 60 minutes), the apparatus 10 can be configured to heat the microfluidics-based biochip 20 to a second temperature (e.g., from about 90° C. to about 100° C., or about 95° C.) to terminate RT-LAMP reactions. Optionally, the microfluidics-based biochip 20 can be held at the second temperature for a predetermined time (e.g., from about 2 minutes to about 30 minutes, or about 5 minutes). The microfluidics-based biochip 20 can then be cooled to a third temperature (e.g., from about 0° C. to about 20° C., or about 4° C.) for a predetermined time (e.g., from about 2 minutes to about 30 minutes, or about 5 minutes).

In some aspects, a computing device 1001 can be configured to detect presence of biomarkers of interest (e.g., FABP3 and/or FABP4) beyond a threshold. For example, a fluorescence above 3000 arbitrary units (AU) can be indicative of an increased risk of PrI. In some aspects, the computing device 1001 can be in communication with the camera 70 or other optical sensor. The computing device 1001 can provide an output upon receiving a signal from the camera or other optical detector that a detected level of biomarker is above a threshold. For example, in some aspects, the sensor 54 can be a camera 70. The computing device 1001 can determine whether a color detected by the camera is beyond a color threshold. The color threshold can comprise a brightness, an intensity of a particular wavelength of light (or range of wavelengths), a chromaticity, and/or a hue or the like. In other aspects, the sensor 54 can be a fluorescence sensor. The computing device can determine a fluorescence detected by the fluorescence sensor is beyond a fluorescence threshold. In other aspects, the sensor 54 can be a pH sensor. The computing device can determine a pH detected by the pH sensor is beyond a pH threshold.

The output can include any suitable output for conveying crossing of the threshold. For example, the output can comprise a visual indication (e.g., a light turning on or changing color), an audible output (e.g., a tone or series of tones or a voice recording). In other aspects, the output can be an output to a display, such as a smartphone, tablet, personal computer, or other computing device. The output on the display can convey any relevant information, such as raw data (e.g., absolute or relative values of the detected level of biomarker of interest), graphical data, or other processed data (e.g., an indication that the at least one biomarker is above a threshold). In further aspects, it is contemplated that combinations of the above-described outputs can be provided concurrently in response to the crossing of a threshold.

In still further aspects, the biomarker(s) of interest can be compared to a plurality of thresholds. Each threshold can correspond to a different risk level. For example, different thresholds can correspond to different risk levels. In one aspect, a first threshold can correspond to a mild risk for PrI, a second threshold can correspond to a moderate risk of PrI, and a third threshold can correspond to a high risk of PrI. In further or alternative aspects, the detected metric indicative of amount of biomarkers of interest can be provided to a clinician in an absolute or relative value (rather than as compared to a threshold), and the clinician can provide a judgment based on the detected metric indicative of the amount of biomarker(s) of interest provided by the apparatus 10. That is, the output of the apparatus 10 need not be a binary output of above or below the threshold(s).

It is contemplated that the threshold(s) for the biomarkers of interest (e.g., FABP3 and/or FABP4) can be a function of a particular individual. Accordingly, the amount of biomarker(s) of interest that is indicative of PrI can be relative to a level of at least one reference gene. In various optional aspects, the reference gene can be Ubiquitin C (UBC), (3-2 Microglobulin (B2M), Actin (ACTIN), Hypoxanthine phosphoribosyltransferase-1 (HPRT1), and/or large ribosomal protein P0 (RPLP0). In some aspects, a method can comprise detecting a relative amount of the biomarker of interest relative to a reference gene of an individual and adjusting the threshold based on normalization to the level of the reference gene present. For example, the reference gene activity can be input into the computing device 1001, and the computing device can adjust the threshold based on the reference gene activity. Optionally, as further disclosed herein, the adjustment to the threshold can be determine by reference to a database concerning information concerning the association between various sensed/detected signals and biomarkers and/or genetic activity.

Accordingly, in some aspects, threshold(s) disclosed herein can be absolute thresholds, corresponding to a measured level of the at least one biomarker indicative of PrI. In alternative aspects, the threshold(s) disclosed herein can be relative thresholds, corresponding to a measured level of the at least one biomarker indicative of PrI relative to a measured level of a reference gene. In still further aspects, the threshold(s) disclosed herein can correspond to a measured level of the at least one biomarker indicative of PrI relative to measured levels of a plurality of reference genes. Accordingly, in some aspects, the computing device 1001 can receive a first measurement corresponding to a level of at least one biomarker indicative of PrI and a second measurement corresponding to a level of a gene. The computing device 1001 can, based on the first and second measurements, provide an output corresponding to a risk level. The output can be, for example, an indication that a ratio of the first measurement to the second measurement is above a relative threshold.

The computing device 1001 can comprise, or be in communication with, a database having information associated with correlating signals from the sensor(s) 54 with levels of the biomarkers to be detected. For example, the database can comprise information reflecting one or more of: associations of sensed brightness with one or more biomarkers; associations of intensity of a particular wavelength of light (or range of wavelengths) with one or more biomarkers, associations of a chromaticity with one or more biomarkers, and/or associations of hue or the like with one or more biomarkers. The database of the computing device 1001 can further comprise any relevant calibration data. For example, it is contemplated that detected fluorescence intensity can be a function of spacing of the sensor from the sample. Accordingly, the computing device 1001 can store calibration data for correlating signals from the sensor(s) 54 with levels of the biomarkers to be detected. Further, the database of the computing device 1001 can store one or more thresholds as disclosed herein. Accordingly, in some optional aspects, the computing device 1001 can recall information from the database in order to: (a) translate the signals from the sensor(s) into a measurement of the biomarker; and/or (b) compare the measurement of the biomarker to a threshold. Optionally, it is contemplated that the information within the database can be associated with various patient populations based on various factors, such as age, condition, sex, height, weight, and the like. Thus, in some aspects, it is contemplated that the determination of the correlation of levels of biomarkers with signals detected by sensors as disclosed herein can be made with reference to data/information associated with patients having one or more shared attributes/factors with the patient being examined/analyzed.

Optionally, the database of the computing device 1001 can comprise a lookup table that permits correlating a detected level of a biomarker of interest relative to a level of a reference gene of an individual to determine a risk of PrI. For example, the computing device can detect a table value corresponding to a level of biomarker of interest and a level of reference gene. Said table value be indicative of PrI risk (e.g., binary yes/no risk, high/low risk, a relative risk, a weighted risk, etc.) In other aspects, the computing device 1001 can store instructions that when executed by a processor, cause the processor to apply a mathematical formula to correlate the detected level of a biomarker of interest relative to a level of a reference gene of an individual to determine a risk of PrI. Optionally, the formula can be a ratio (e.g., level of biomarker divided by level of reference gene). Said ratio can optionally be compared to a threshold ratio.

In some aspects, the microfluidics-based biochip can be configured to perform an RT-LAMP process to detect the respective level of the at least one biomarker. In exemplary aspects, the microfluidics-based biochip can be configured to measure a plurality of biomarkers concurrently.

In some optional aspects, the microfluidics-based biochip 20 can comprise a laminated-microdevice fabrication process with biocompatible, chemically inert substrate (e.g., comprising polymethyl methacrylate (PMMA)) plastic and cap. Electrodes (e.g., gold electrodes) can be screen printed onto the first and second layers 30, 32.

In exemplary aspects, the microfluidics-based biochip 20 can have a size of 26 mm×9 mm×3 mm and a total sample volume of 9 μL.

The microfluidics-based biochip 20 can be disposable. The apparatus 10 can be configured to process a small amount of whole blood (e.g., <10 μL).

Upon determining, by the apparatus 10, that a particular individual is at an elevated risk for PrI, a clinician can prescribe certain remediating strategies. For example, more cushioning can be prescribed or provided (e.g., for a wheelchair or for a bed). In further aspects, an automated weight-shifting cushion can be prescribed. In still further aspects, an implanted neuromuscular electrical stimulation (NMES) system can be prescribed to provide automated weight-shifting and improve tissue health.

### Biomarkers for Recurrent Pressure Injury Risk

While all persons with SCI are at increased risk of PrI development, susceptibility for PrI as a secondary complication can be unique for each individual. It can be shown that the gluteal muscles for persons with SCI are highly heterogeneous and frequently highly infiltrated with adipose tissue. Differences in gluteal muscles are not clearly associated with either level of injury or AIS grade. Changes in soft tissue composition and function following SCI can provide insight to personalized risk status. A clinician can use this insight to determine each individual's optimal PrI prevention regime.

Many biomarkers to identify individuals at risk for specific diseases. Network topology analysis shows that small tissue samples can be representative of the whole body metabolism. Collection of a biological sample to determine diagnostic biomarkers can minimally invasive techniques. For example, blood samples can optionally be used instead of a tissue biopsy. However, a correlative relationship between circulatory and local biomarkers can be established. When considering PrI risk, circulatory biomarkers indicative of soft tissue characteristics at the gene expression level can be identified that are relate to increased propensity for recurrent PrI.

Intracellular fatty acid-binding proteins (FABPs) are advantageous candidates for PrI risk biomarkers because they are present in nearly all tissues and the majority of FABPs show high affinity for specific tissue types, although some can be found in more than one type. Of particular relevance to PrI development, FABP4 can be expressed highly in adipose tissue, while in muscle the major FABP is FABP3 (also known as H-FABP). FABP4 can be expressed in muscle. However, there is a magnitude difference in expression levels between FABP3 and the much lower expression of FABP4 in the muscles of highly trained individuals. This difference can be attenuated in individuals with SCI who have dystrophic-type changes in muscle quality. It was found that FABP3 was increased in one individual with SCI who had an active PrI. It was further found that circulatory levels of both FABP4 and FABP3 are significantly correlated (p<0.0001) with recurrent PrI over time.

### Detection of Biomarkers

Reverse transcription polymerase chain reaction (RT-PCR) is the “gold-standard” laboratory-based method to measure RNA expression of circulatory biomarkers. However, this method is costly, time-consuming, and typically only available in a large reference laboratory. For example, a typical RT-PCR machine costs tens of thousands of dollars and a single plate for analysis can easily cost $3,000-$5,000. Typical processing time is on the region of 6-8 hours for sample preparation and thermal cycling.

Reverse transcription loop-mediated isothermal amplification (RT-LAMP) offers a practical alternative to RT-PCR as it can be easily deployed outside the reference laboratory. RT-LAMP can be carried out at a low constant temperature (e.g., about 65° C.) in one step, obviating the need for specialized and expensive thermal cycling equipment in RT-PCR. Rapid and efficient amplification of a target RNA sequence from minimally processed samples can be achieved through the use of four primers targeting six regions of the target sequence, with RNA being amplified 10⁹-10¹¹ times in 15-60 min. LAMP-based molecular detection has been shown to have sensitivity and specificity comparable to, or exceeding that of PCR, and real-time turbidimetric or colormetric readout methods offer rapid and quantitative assessment with minimal instrumentation. The isothermal nature of the RT-LAMP method combined with simple and low-complexity instrumentation for readout are ideal characteristics for integration into a low-cost, disposable microfluidic chip as part of portable platform for molecular detection of circulatory biomarkers. Furthermore, real-world deployment of LAMP-based assays have recently been shown to be useful for the detection of malaria and tuberculosis in resource limited settings. It was determined that a rapid and cost effective test for PrI risk that can be performed outside of the reference laboratory to provide a point-of-care test that can be performed by nurse or a patient in a home or clinic setting.

### Exemplary Demonstrations

Exemplary demonstrations as disclosed herein showed linkages between muscle tissue biomarkers and tissue resilience under applied loads in individuals with SCI. The demonstrations addressed the conundrum of why many suffer from a continuous cycle of recurring PrI and delayed rehabilitation while others remain pressure injury free.

It is contemplated that alteration in muscle tissue composition can be a critical objective indicator of increased pressure ulcer susceptibility in individuals with SCI. Detailed analysis of gluteal muscle characteristics and tissue health responses under physiological loading conditions can provide a personalized indication of risk status enabling the clinician to determine an individual's optimal PrI prevention regime following SCI. The exemplary demonstration identified individual PrI risk characteristics following SCI as a basis for development of a clinical tool to optimize personalized pressure relief which recognizes that every active duty military and veteran with SCI is an individual. Maintenance of tissue health provides a foundation for all persons with SCI to achieve optimal outcomes from their initial rehabilitation which can enable them to maximize their quality of active life.

### Evaluation of Gluteal Muscle Composition and Tissue Health Under Physiologically Relevant Loading Conditions

Details of gluteal muscle composition in individuals with SCI and able-bodied controls were initially examined using pelvic computed tomography (CT) with contrast. It was found that gluteal muscle characteristics indicative of impaired tissue viability were present in individuals with SCI. SCI disuse muscle atrophy was anticipated. Analysis further indicated that intramuscular atrophy was not uniform. In addition, SCI muscle composition showed increased proportions of both low density muscle and intramuscular fat infiltration. Multivariate assessment methodology was applied to assess unloaded tissue health in some of these individuals. Specifically, SCI muscle composition showed increased proportions of both low density muscle and intermuscular adipose tissue (IMAT). Subsequent repeated measures observational study has examined relationships between health factors, health behaviors, and tissue health together with CT-based evaluation of gluteal muscle composition.

A repeated measures study of 38 individuals with SCI has been carried out.

TABLE 1
Participant clinical factors

| | | N | % |
|---|---|---|---|
| Neurological | Upper motor neuron | 27 | 71% |
| involvement | Lower motor neuron | 11 | 29% |
| AIS Grade | Complete (AIS A) | 10 | 26% |
| | Incomplete (B, C D) | 28 | 74% |
| Pressure | Yes | 27 | 71% |
| Injury history | No | 11 | 29% |

Exclusion criteria included having an open pelvic region PrI at the time of recruitment, presence of a systemic disease or condition that is known to impact inflammatory biomarkers or PrI risk, e.g. uncontrolled diabetes or heart disease, and known sensitivity to intravenous (IV) contrast. At intake, a comprehensive profile of clinical and health factors was obtained together with demographic information relevant to PrI history. Study participants were 22-80 years old at enrollment (mean 58 years) and had sustained SCI 1 month-46 years prior to entering the study (mean 9 years). Clinical factors were representative of Veterans with SCI (Table 1). Tissue health assessment and pelvic CT scans were carried out at recruitment and repeated annually for the duration of participation.

Pelvic CT scans with contrast were obtained using a Philips Brilliance 16 CT system (Philips Medical Systems, Cleveland, OH) by a single qualified radiology technologist under the supervision of expert radiologists. All scans were performed in the supine position with a cushion placed under the legs to minimize soft tissue compression in the buttocks. To limit radiation exposure, an initial anterior-posterior scout image of the pelvis was used to define the limits of the scan. Iodinated contrast agent was administered intravenously in two half-dose 50 cc boluses at 80 seconds and 40 seconds before the scan followed by saline, allowing both the arteries and the veins to display contrast.

Axial images of the gluteus maximus muscle with a 0.4 mm slice thickness were obtained from the superior origin near the sacrum to an inferior point below the ischial tuberosities (IT). The gluteal muscle region of interest (ROI) was identified by outlining the fascia of the gluteus maximus muscle in each axial CT image slice using Amira (Visualization Sciences Group, Burlington, MA). This ROI was defined as the muscle cross-sectional area (CSA) in each slice. Overall muscle volume was then calculated by multiplying CSA by slice thickness for all slices. Intramuscular fat infiltration of the gluteal muscle was determined by selection of axial muscle slices for further analysis using ImageJ. These cross-sections were located at level A, the midpoint of the S2/S3 sacral vertebrae, level B, the superior margin of the greater trochanters (GT) and level C, the inferior margin of the ischial tuberosities (IT).

X-ray attenuation in CT scans is influenced by the density of the material being imaged. Tissues with higher density, such as lean muscle, appear brighter than lower density tissue, such as adipose regions. This attenuation is quantified using the linear transformation Hounsfield Unit (HU) scale which was derived for use in soft tissue imaging. The scale is calibrated using air at the minimum of −1024 HU with distilled water as baseline at 0 HU and bone at around 400 to 1000 HU. The use of contrast increased the brightness of the vasculature such that the HU value was the same or higher than lean muscle. Overall muscle composition was determined by calculating the ratio of pixels within the specified range for each tissue type of interest to the total pixels in the muscle CSA.

Tissue health assessments were carried out in unloaded and seated postures. All study participants used their own wheelchair cushion which had been prescribed by the Louis Stokes Cleveland Veterans Affairs Medical Center (LSCVAMC) SCI/D Seating and Mobility Clinic. Responses were quantified in real time under physiologically appropriate loads using a Tissue Health Evaluation Toolbox (THEToolbox), a standardized modular non-invasive, multivariate methodology for assessment of tissue health status. THEToolbox includes monitoring of interface pressure (IP) and transcutaneous oxygen pressure (TcPO₂).

Data analysis: The comprehensive multivariate study design allowed intergroup differences in proportions of at least 0.30 between groups to be determined using a Fisher's exact test with a power of 0.9. For continuous and categorical variables, the application of more comprehensive statistical models allowed detection of smaller group differences with the same power. Study data did not have normal distribution; therefore, non-parametric statistics were used to compare tissue health and gluteal muscle composition variables between persons with and without a PrI history.

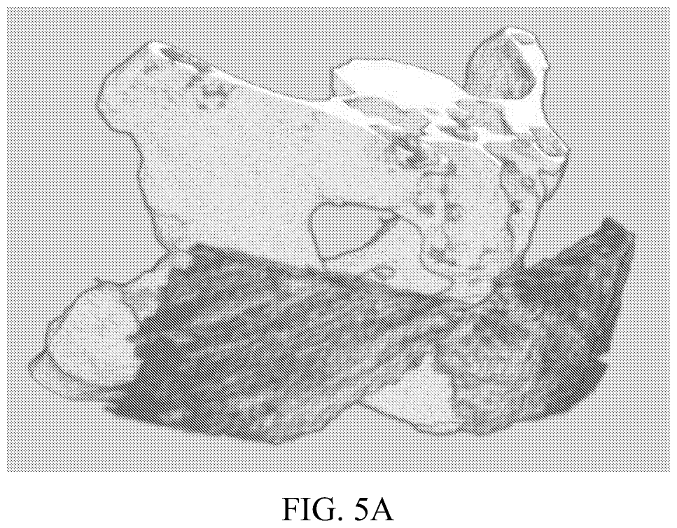

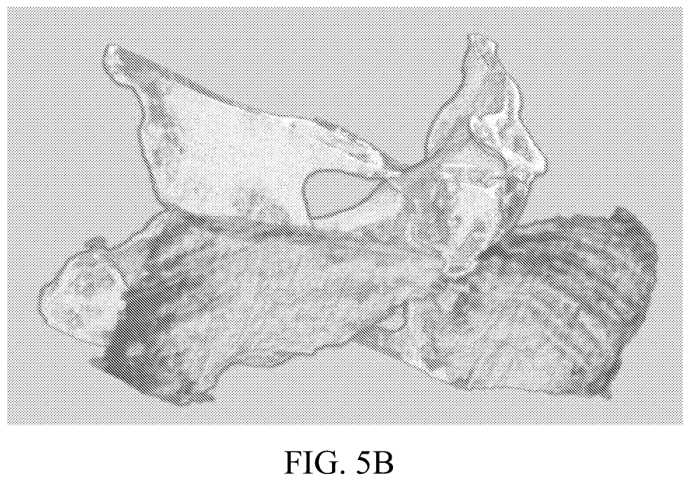

CONFORMat® proprietary software creates 2D/3D IP distribution maps which graphically display sensor mat data, measured at 2 Hz, producing a 400 frame data set for analysis. These data were exported as numerical arrays and recreated in Matlab for further analysis. Pressure data arrays were aligned and standard ROI around each ischium were identified. The ischial tuberosity analysis ROI were bilateral 5×5 sensel regions (approximately 7.5×7.5 cm²). Mean IPs within each analysis ROI were determined for each assessment phase. It was found that mean seated ischial region interface pressures for individuals with or without a PrI history was the same, as shown in FIG. 5. As Gefen et al. previously reported using a physical model evaluation, internal forces cannot be predicted from surface IP measurement. Surface IP be can similar while internal mechanics change due to higher IMAT which compromises tissue resilience.

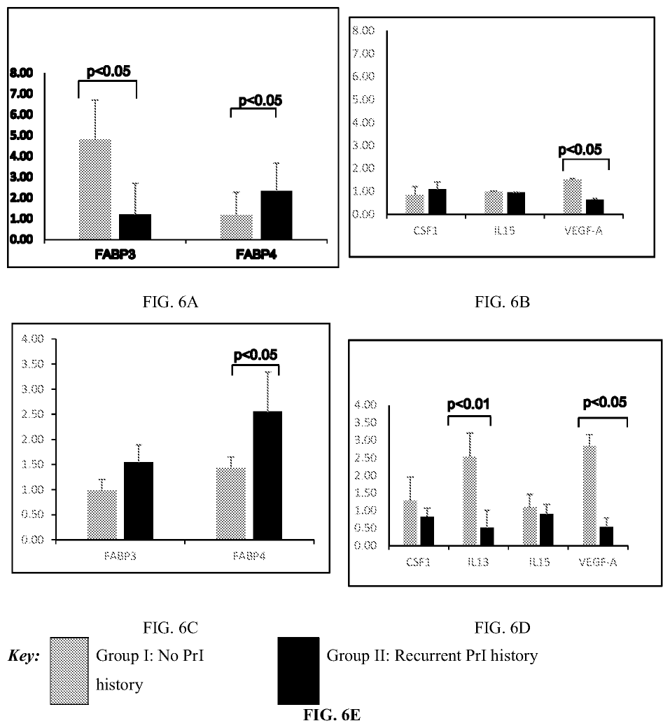

In looking at initial and follow up evaluations of muscle composition, it was found that intramuscular adipose tissue (IMAT) levels varied considerably even between individuals with similar AIS grade or level of injury. For some individuals, muscle tissue in the gluteal muscle region has been almost entirely replaced by IMAT. It was also found that while IMAT is stable over time for many individuals, varying by only 0-5%, it can change dramatically for others, even several years post-injury, as shown in the changes from FIG. 6A to FIG. 6B.

Body habitus does not appear to be a factor. Several persons in the exemplary study cohort with very high levels of IMAT had low to normal body habitus. Changes in IMAT were also not consistently related to age or AIS grade in the current study cohort. During the 3 year study follow up, 20% of the study cohort developed one or more PrI. All individuals were in the high risk, high IMAT, group.

In summary, detailed analysis of gluteal muscle composition and tissue health responses under physiologic loading provides some insight into hidden tissue health factors impacting personalized risk status. The preliminary work found that interface pressure alone is an inadequate measure of PrI risk. There was no difference between mean ischial pressure values for persons with or without a PrI history. IMAT is a clinically significant biomarker of PrI risk. Individuals with greater than 15% gluteal IMAT were statistically highly significantly (p<0.001) more likely to have a history of severe or recurrent PrI.

Muscle Composition Associations with Circulatory and Local Fatty Metabolite Activity:

Biomarkers for recurrent pressure injury risk in persons with spinal cord injury: It was further studied whether changes in IMAT are also expressed by changes in muscle-based and circulatory biomarkers. Specifically, potential linkages between fatty metabolite and inflammatory biomarkers and PrI recurrence following SCI were examined.

The study cohort comprised 30 individuals with complete or incomplete SCI. Study participants either had never developed a PrI (Group I) or had a history of recurrent PrI (Group II). Gluteal muscle biopsy and whole blood samples were obtained annually over a 2-3 year period concurrent with muscle composition and tissue health assessments.

Whole blood concentrations of circulatory biomarkers were determined from fasting blood samples, and snap-frozen in liquid nitrogen before storage at −80° C. until processing using qPCR and Western blotting. Muscle biopsies were collected to determine localized biomarkers from the gluteus maximus under CT guidance using an 18-gauge Temno coaxial core biopsy system (CareFusion, San Diego, CA). Biopsy tissue was collected under local anesthesia (1% lidocaine), then snap-frozen, stored, and processed as for whole blood samples.

RNA was extracted from muscle and whole blood samples using the QIAzol Lysis Reagent, Qiagen Mini Kit, and Qiagen Micro Kit (Qiagen, Valencia, CA) following standard procedures. Gene expression was measured by qRT-PCR for over 100 genes involved in fatty metabolism and muscle damage/inflammation using commercially available custom RT2 Profiler PCR array plates. RNA expression levels were followed up with Western blotting to confirm expression differences at the protein level.

A 50% detection rate was used to identify biomarkers for which calibrated relative normalized quantities (CRNQ) were reliably reported in blood and muscle samples. The CRNQ data was log-transformed by qbase+, thus any skewed raw data approximately conformed to normal distribution. The F-test was run to determine whether variance was equal between groups. The t-test for samples of unequal variance was then applied as appropriate. Effect size was determined using Hedge's g. Relationships between CRNQ levels of biomarkers of interest and IMAT were also determined.

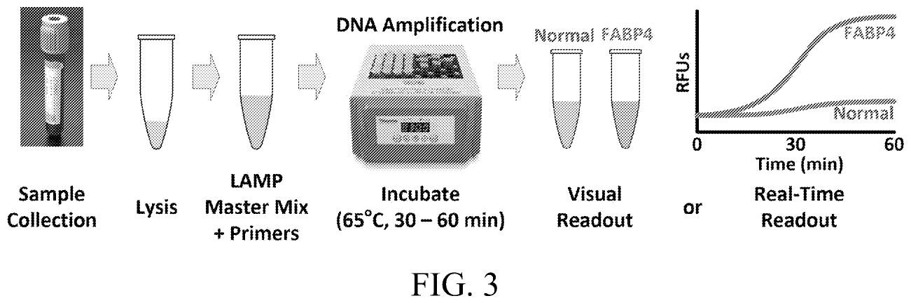

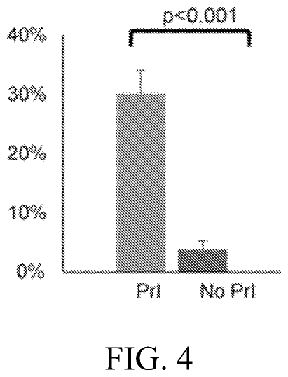

The primary fatty metabolite biomarkers of interest in the muscle and blood samples were FABP4, predominant in adipose tissue, and FABP3, predominant in skeletal muscle. Fatty metabolite biomarkers were reliably detected in both muscle and blood samples for both Groups. CRNQ levels for FABP4 were significantly higher in both muscle (p<0.05, effect size=0.85) and blood (p<0.05, effect size=1.41) for persons in Group II (those with recurrent PrI history), illustrated in FIG. 3 as the black bar. FABP3 was significantly higher in muscle samples for persons in Group I (p<0.05, effect size=0.94). Circulatory FABP3 levels were lower for Group I, although this did not reach statistical significance. Both FABP3 and FABP4 were highly significantly correlated with IMAT in both muscle and blood samples (p<0.001, effect size>0.9, for all comparisons).

Overall, inflammatory biomarkers were more reliably detected in the blood compared to muscle samples. CRNQ levels for inflammatory biomarkers in muscle were lower than for fatty metabolites. Both CSF-1 and IL-15 were found in muscle and blood samples at similar levels for both groups. CSF-1 was slightly higher in Group II muscle samples but not in blood samples (FIG. 6C), while IL-15 was slightly higher in blood samples. These differences did not reach significance. However, IL-13 was significantly higher (p<0.01, effect size=1.05) in the blood of persons in Group I (FIG. 6C), but did not meet the detection rate threshold for muscle samples in the study cohort. VEGF-A was significantly increased in Group I muscle (p<0.05, effect size=0.71) and blood (p<0.05, effect size=0.63) samples, with slightly higher expression levels in blood (FIG. 6D).

In summary, the levels of inflammatory biomarkers observed were lower than for fatty metabolites of interest The exclusion criteria required that no study participant had an active inflammatory disease or uncontrolled diabetes. Thus although statistically significant it is unclear whether these difference have a clinical significance without consideration of other factors.

However, circulatory biomarkers of fatty metabolism, specifically FABP4 and FABP3, show high levels of expression, are reliably detected and are statistically different between persons with or without a history of recurrent PrI.

Thus findings indicate that circulatory levels of FABP4 and FABP3 hold great potential as a recurrent PrI propensity biomarkers. RT-PCR analysis of circulatory biomarkers of interest in whole blood lays the foundation for development of a blood test for recurrent PrI propensity. However, the approaches used in the Exemplary Demonstration above used expensive laboratory-based equipment and requires hours to days to fully process.

### Development in Microfluidic Platforms for Point of Care (POC) Patient Health Monitoring

Microfluidic dielectric sensor for POC monitoring of PrI indicators: A major point of novelty in the systems and methods disclosed herein is the development of a new microfluidics-based biochip technology that can enable widespread, consistent, reproducible, and standardized analysis of PrI risk. In some aspects, and as further disclosed herein, it is contemplated that the disclosed devices and methods can permit measurement of levels of multiple biomarkers concurrently.

### Objective of Disclosed Technology

PrI prevention following SCI remains challenging. Good risk assessment tools to reliably identify individuals with SCI with higher propensity for recurrent PrI were not available. There was currently no straightforward method for sensitively screening fresh blood as a POC test to detect the biomarkers of interest. A portable and disposable, pressure injury propensity biosensor, a POC diagnostic device, is disclosed. The POC diagnostic device can enable an individual's PrI propensity to be determined by a blood test that can be administered by the clinician or even the individual at home without the need to process samples and extract plasma prior to analysis, in the same way as persons with diabetes check their blood sugar levels routinely.

The POC diagnostic device can be a microfluidic biochip for recurrent PrI propensity based on a novel RT-LAMP platform. The ultimate research goal is to validate a home blood test that can be administered by the patient, caregiver or a nurse. The ability to test personal risk status at home can reduce the travel burden for many individuals at risk, including Veterans who live hours from their nearest VA Medical Center.

### Specific Features

A major risk factor in biomechanical aspects of PrI prevention has been overlooked: the soft tissues being loaded are not the same for every individual at risk. Furthermore, these changes in muscle composition are detectable in circulatory biomarkers. The POC diagnostic device can provide develop a benchtop low-cost, simple, and rapid blood test based on RT-LAMP for assessment of PrI risk that can be performed outside of a central laboratory. The disclosed POC diagnostic device can enable risk testing to be administered by the clinician or even the individual at home without the need to process samples and extract plasma prior to analysis, in the same way as persons with diabetes can check their blood sugar levels routinely.

Stage 1: Design of RT-LAMP Primers for FABP3 and FABP4 and Verification of RT-LAMP with Clinical Samples.

### RT-LAMP Analysis can Provide Rapid Low-Cost Analysis of Circulatory Biomarkers for Recurrent PrI Risk

A lab-based blood test based on RT-LAMP can be configured for the rapid detection of circulatory biomarkers FABP3 and FABP4. Verification in a lab-based RT-LAMP assay can be carried out using existing samples from the preliminary study. Blood samples for Veterans with SCI known to have high and low FABP4 and FABP3 can be selected from over 200 stored samples. RT-PCR analysis has been carried out for these samples. PrI history and muscle composition is also known. Validation can be carried out using 20 de novo blood samples, which can be obtained in other ongoing studies.

### Stage 2: Development of the Microfluidics-Based Biochip, a Novel Portable Multiplexed RT-LAMP Microsensor for Recurrent PrI Propensity Detection.

A RT-LAMP Microsensor can have the Same Performance as a Lab-Based System

The microfluidic biochip for RT-LAMP-based detection of circulatory biomarkers FABP3 and FABP4 for assessment of recurrent PrI propensity is disclosed. Validation of the RT-LAMP microsensor can include comparison with RT-PCR and lab-based RT-LAMP analysis using existing blood samples. Performance outcomes measures can include measurement accuracy, time to process samples and output readability.

### Methods

Specific Stage 1: Design of RT-LAMP Primers for FABP3 and FABP4 and Verification of RT-LAMP with Clinical Samples

RT-LAMP primers can be designed for FABP3 and FABP4 using Prime Explorer V4 [i]. A set of four RT-LAMP primers can be designed targeting six regions of the sequence with a target melting temperature (Tₘ) of 60-65° C. FIG. 3 shows the workflow of the RT-LAMP test: Whole blood samples can be transferred (e.g., pipetted) into a tube containing lysis buffer (QIAzol Lysis Reagent, Qiagen, Valencia, CA) to release RNA. 10 μL of the lysed blood sample can then be added to a mixture containing 12.5 μL of 2× WarmStart Colorimetric LAMP Master Mix (New England BioLabs, Cat #M1800S) and 2.5 μL of LAMP primer mix. The tube can be sealed and then incubated in a dry block heater (Thermo Fisher Scientific) to allow for the amplification phase to take place. The required time for the amplification phase can be determined in the design of the LAMP primers and is expected to take place with 30-60 minutes. The readout of the assay can be visual observation of a color change that take place in the presence of amplified RNA.

TABLE 2
Fatty metabolite blood sample distribution

| | High | Low |
|---|---|---|
| FABP4 | 10 | 10 |
| FABP3 | 10 | 10 |

Verification with clinical samples: RT-LAMP assay sensitivity can be determined using serial dilutions of the target biomarkers (FABP3 and FABP4). Verification of the RT-LAMP assay using clinical samples can be carried out using existing samples from an exemplary preliminary demonstration. A balanced selection of blood samples for Veterans with SCI known to have high and low FABP4 and FABP3 can be selected from over 65 stored samples (see Table 2). Whole blood concentrations of circulatory fatty metabolite biomarkers have been determined from fasting blood using RT-PCR analysis for these samples. PrI history and muscle composition is also known.

RT-LAMP assay validation: External validation of the RT-LAMP assay can be carried using 20 de novo blood samples, which can be obtained in other ongoing studies at LSVAMC. RT-PCR analysis of FABP4 and FABP4 can be carried out to provide compassion with RT-LAMP outputs. A comprehensive de-identified profile can be collected including demographics, clinical factors related to SCI, health factors.

If the RT-LAMP analysis of FABP4 and FABP3 biomarkers does not provide sufficient sensitivity to predict muscle composition or PrI occurrence. If FABP4 and FABP3 biomarkers are alone insufficient, additional circulatory biomarkers (CSF-1, IL-13, etc.) can be detected.

### The Microfluidic Biochip, a Novel Portable Multiplexed RT-LAMP Microsensor for Recurrent PrI Propensity Detection.

The microfluidic biochip biosensor can comprise a microfluidic analysis chamber to perform the RT-LAMP reaction. Microfluidic system design and fabrication is well established in the laboratories of Co-Is Gurkan, Suster and Mohseni, and is based on a low-cost (material cost of <$1 per chip), laminated-microdevice process that has been used as the platform for the ClotChip and HemeChip microsensors (see Preliminary Studies). Biomedical-grade PMMA (McMaster-Carr) can be used, which allows for ease of fabrication, low cost and disposable use, which are important features for POC sensor development.

The microfluidic biochip biosensor (FIG. 1A) comprises three parts, namely, a bottom PMMA substrate, a top PMMA cap and a double-sided-adhesive (DSA) film that forms the microfluidic channel and bonds the top and bottom PMMA layers together. Two microfluidic analysis chambers can be fabricated on a single chip to allow for simultaneous detection to two target biomarkers. microfluidic biochip can be fabricated using a laser micromachining platform (Versa Laser VLS 2.3) to create the inlet, outlet, and fluidic channels. Channel dimensions are controlled to within 10 μm with this fabrication method, which ensures reliability and repeatability. If needed, the components can be sterilized by ultraviolet (UV) light and assembled in a sterile, laminar-flow hood (EdgeGard, The Baker Company).

Experimental procedure: As shown in FIG. 2, the microfluidic biochip can be mounted on an indium tin oxide (ITO) heater for thermal control. The optically transparent ITO heater allows for real-time detection of colorimetric or fluorescent changes of the sample in the analysis chamber using a low-cost camera. The RT-LAMP primers and reagents for the microfluidic biochip experiments can be the same as in the RT-LAMP experiments in Stage 1. Images captured during the experiment can be analyzed with MATLAB computational software (Mathworks) to quantify colorimetric changes in the sample and a threshold that indicates the presence of the biomarker can be determined.

Device verification: Sensitivity of the microfluidic biochip can be determined using serial dilutions of the target biomarkers (FABP3 and FABP4). Clinical samples used in Stage 1 can also be used for verification of microfluidic biochip performance. Twenty blood samples from persons with SCI known to have high or low levels FABP4 and FABP3 can be tested with the microfluidic biochip microsensor. the clinical detection sensitivity can be compared to that of RT-PCR from the exemplary demonstrations and that of RT-LAMP from experiments in Stage 1.

Device performance: Measurement accuracy, time to process samples and output readability can be evaluated. The target specifications are for the microfluidic biochip to provide accurate results in under 20 minutes, with visual outputs that clearly distinguish between high and low levels for both FABP4 and FABP3.

In aspects in which colorimetric changes in the small sample volume are not be sufficient to detect with a low-cost camera, fluorescent readout method can be used at the expense of increased complexity and cost associated with UV light excitation source.

### Exemplary Computing Device

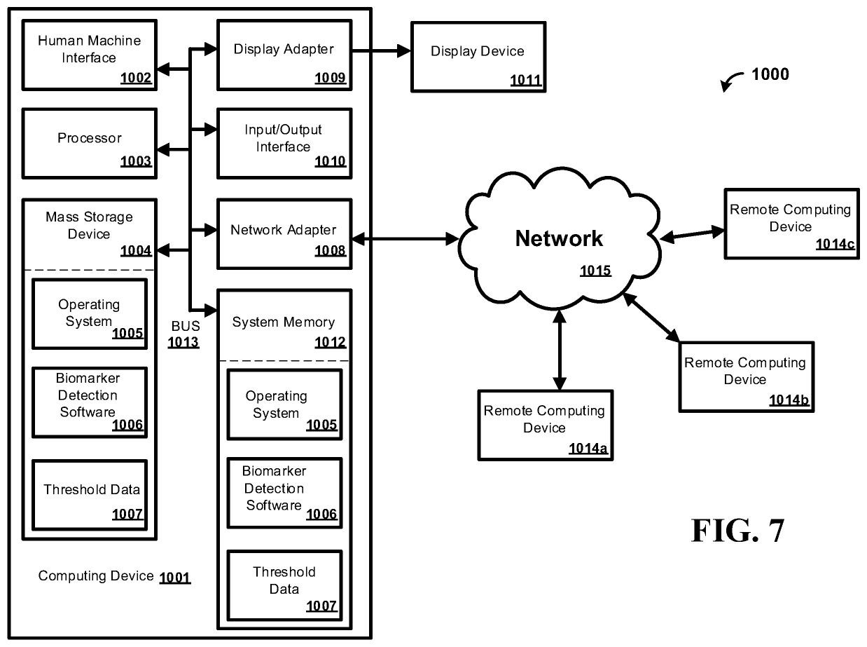

FIG. 7 shows an exemplary operating environment 1000 including an exemplary configuration of a computing device 1001 for use with the apparatus 10 (FIG. 1A). In various aspects, the computing device 1001 can embodied as one or more separate computing devices (e.g., optionally, microcontrollers).

The computing device 1001 may comprise one or more processors 1003, a system memory 1012, and a bus 1013 that couples various components of the computing device 1001 including the one or more processors 1003 to the system memory 1012. In the case of multiple processors 1003, the computing device 1001 can utilize parallel computing.

The bus 1013 may comprise one or more of several possible types of bus structures, such as a memory bus, memory controller, a peripheral bus, an accelerated graphics port, and a processor or local bus using any of a variety of bus architectures.

The computing device 1001 may operate on and/or comprise a variety of computer readable media (e.g., non-transitory). Computer readable media may be any available media that is accessible by the computing device 1001 and comprises, non-transitory, volatile and/or non-volatile media, removable and non-removable media. The system memory 1012 has computer readable media in the form of volatile memory, such as random access memory (RAM), and/or non-volatile memory, such as read only memory (ROM). The system memory 1012 may store data such as threshold data 1007 and/or program modules such as operating system 1005 and biomarker detecting software 1006 that are accessible to and/or are operated on by the one or more processors 1003.

The computing device 1001 may also comprise other removable/non-removable, volatile/non-volatile computer storage media. The mass storage device 1004 may provide non-volatile storage of computer code, computer readable instructions, data structures, program modules, and other data for the computing device 1001. The mass storage device 1004 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1004. An operating system 1005 and biomarker detecting software 1006 may be stored on the mass storage device 1004. One or more of the operating system 1005 and biomarker detecting software 1006 (or some combination thereof) may comprise program modules and the biomarker detecting software 1006. The threshold data 1007 may also be stored on the mass storage device 1004. The threshold data 1007 may be stored in any of one or more databases known in the art. The databases may be centralized or distributed across multiple locations within the network 1015.

A user may enter commands and information into the computing device 1001 using an input device. Such input devices comprise, but are not limited to, a joystick, a touchscreen display, a keyboard, a pointing device (e.g., a computer mouse, remote control), a microphone, a scanner, tactile input devices such as gloves, and other body coverings, motion sensor, speech recognition, and the like. These and other input devices may be connected to the one or more processors 1003 using a human machine interface 1002 that is coupled to the bus 1013, but may be connected by other interface and bus structures, such as a parallel port, game port, an IEEE 1394 Port (also known as a Firewire port), a serial port, network adapter 1008, and/or a universal serial bus (USB).

A display device 1011 may also be connected to the bus 1013 using an interface, such as a display adapter 1009. It is contemplated that the computing device 1001 may have more than one display adapter 1009 and the computing device 1001 may have more than one display device 1011. A display device 1011 may be a monitor, an LCD (Liquid Crystal Display), light emitting diode (LED) display, television, smart lens, smart glass, and/or a projector. In addition to the display device 1011, other output peripheral devices may comprise components such as speakers (not shown) and a printer (not shown) which may be connected to the computing device 1001 using Input/Output Interface 1010. Any step and/or result of the methods may be output (or caused to be output) in any form to an output device. Such output may be any form of visual representation, including, but not limited to, textual, graphical, animation, audio, tactile, and the like. The display 1011 and computing device 1001 may be part of one device, or separate devices.

The computing device 1001 may operate in a networked environment using logical connections to one or more remote computing devices 1014a,b,c. A remote computing device 1014a,b,c may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. The remote computing devices 1014a,b,c, can perform respective operations of the system. For example, one remote computing device 1014a can be a controller of an AGV. One remote computing device 1014b can control a winding machine. Logical connections between the computing device 1001 and a remote computing device 1014a,b,c may be made using a network 1015, such as a local area network (LAN) and/or a general wide area network (WAN), or a Cloud-based network. Such network connections may be through a network adapter 1008. A network adapter 1008 may be implemented in both wired and wireless environments. Such networking environments are conventional and commonplace in dwellings, offices, enterprise-wide computer networks, intranets, and the Internet. It is contemplated that the remote computing devices 1014a,b,c can optionally have some or all of the components disclosed as being part of computing device 1001. In various further aspects, it is contemplated that some or all aspects of data processing described herein can be performed via cloud computing on one or more servers or other remote computing devices. Accordingly, at least a portion of the system 1000 can be configured with internet connectivity.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A method comprising:
detecting, in a biological sample, using reverse transcription loop-mediated isothermal amplification (RT-LAMP), a respective level of at least one biomarker associated with pressure injuries.

Aspect 2: The method of aspect 1, wherein the at least one biomarker is fatty acid binding protein-3 (FABP3) or fatty acid binding protein-4 (FABP4).

Aspect 3: The method of aspect 2, wherein detecting, in the biological sample, the respective level of the at least one biomarker associated with pressure injuries comprises detecting the respective levels of FABP3 and FABP4.

Aspect 4: The method any one of the preceding aspects, wherein the biological sample comprises blood.

Aspect 5: The method of aspect 4, wherein the blood is whole blood.

Aspect 6: The method of aspect 4 or aspect 5, where the biological sample comprises less than 10 μL of blood.

Aspect 7: The method of any one of the preceding aspects, wherein the at least one biomarker is Kruppel-like factor 4, resistin, cyclin D1, sirtuin 2, dysferlin 2B, pyruvate, dehydrogenase kinase-4, dystrophin, or adiponectin.

Aspect 8: The method of any one of the preceding aspects, wherein the biological sample comprises saliva or urine.

Aspect 9: The method of any one of the preceding aspects, further comprising comparing the respective level of the at least one biomarker to a threshold.

Aspect 10: The method of aspect 9, wherein the threshold is selected based on a level of the at least one biomarker relative to a level of a reference gene.

Aspect 11: The method of aspect 10, further comprising detecting the level of the reference gene.

Aspect 12: The method of aspect 11, wherein detecting the level of the reference gene comprises detecting the level of the reference gene using reverse transcription loop-mediated isothermal amplification (RT-LAMP) concurrently with detecting the respective level of the at least one biomarker associated with pressure injuries.

Aspect 13: The method of any one of the preceding aspects, wherein detecting, in the biological sample, the respective level of the at least one biomarker associated with pressure injuries comprises using a microfluidics-based biochip to detect the respective level of the at least one biomarker associated with pressure injuries.

Aspect 14: The method of aspect 13, wherein using the microfluidics-based biochip to detect the respective level of the at least one biomarker associated with pressure injuries comprises detecting a colorimetric or fluorescence intensity output by the microfluidics-based biochip.

Aspect 15: The method of aspect 13 or aspect 14, wherein using a microfluidics-based biochip to detect the respective level of the at least one biomarker associated with pressure injuries comprises using an apparatus comprising:
the microfluidics-based biochip, wherein the microfluidics-based biochip defines a channel, wherein the microfluidics-based biochip comprises at least one reagent within the channel that includes primers for performing RT-LAMP; and
at least one sensor that is configured to provide a signal indicative of the respective level of the at least one biomarker associated with pressure injuries.

Aspect 16: The method of aspect 15, wherein the at least one sensor comprises a colorimetric sensor.

Aspect 17: The method of aspect 16, wherein the colorimetric sensor is a camera.

Aspect 18: The method of aspect 16 or aspect 17, wherein a color detected by the colorimetric sensor corresponds to the respective level of the at least one biomarker associated with pressure injuries.

Aspect 19: The method of any one of aspects 15-18, wherein the apparatus further comprises a UV source, wherein the at least one sensor comprises a fluorescence sensor, wherein an intensity of fluorescence detected by the fluorescence sensor corresponds to the respective level of the at least one biomarker associated with pressure injuries.

Aspect 20: The method of aspect any one of aspects 15-19, wherein the apparatus further comprises a computing device in communication with the at least one sensor, wherein the computing device is configured to provide an output in response to receiving a signal from the sensor that is above a threshold.

Aspect 21: The method of any one of aspects 15-20, wherein the apparatus further comprises a computing device in communication with the at least one sensor, wherein the computing device is configured to:
receive a first signal indicative of the respective level of the at least one biomarker associated with pressure injuries;
receive a second signal indicative of the respective level of a reference gene; and
provide an output indicative of whether a ratio of the respective level of the at least one biomarker associated with pressure injuries to the respective level of a reference gene is above a relative threshold.

Aspect 22: An apparatus comprising:
a microfluidics-based biochip, wherein the microfluidics-based biochip is configured to detect, in a biological sample, a respective level of at least one biomarker associated with pressure injuries.

Aspect 23: The apparatus of aspect 22 wherein the at least one biomarker comprises FABP3 or FABP4.

Aspect 24: The apparatus of aspect 22 or aspect 23, wherein the microfluidics-based biochip is configured to perform an RT-LAMP process to detect the respective level of the at least one biomarker.

Aspect 25: The apparatus of any one of aspects 22-24, wherein the apparatus comprises an assembly comprising a first layer, a second layer, and an intermediate layer disposed between the first and second layers, wherein the intermediate layer defines a cutout that at least partly defines a channel within the laminated assembly.

Aspect 26: The apparatus of any one of aspects 22-24, further comprising a fluorescence or colorimetric sensor that is configured to provide a signal indicative of the respective level of the at least one biomarker associated with pressure injuries.

Aspect 27: The apparatus of aspect 26, further comprising a computing device in communication with the fluorescence or colorimetric sensor, wherein the computing device is configured to provide an output in response to receiving a signal from the fluorescence or colorimetric sensor that is indicative of the respective level of the at least one biomarker being above a threshold.

Aspect 28: The apparatus of aspect 27, wherein the computing device is configured to:
receive an input of a level of a reference gene present in the sample; and
set the threshold based on the level of the reference gene present.

Although the foregoing invention has been described in some detail by way of illustration and example for purposes of clarity of understanding, certain changes and modifications may be practiced within the scope of the appended claims.

1. A method comprising:
detecting, in a biological sample, using reverse transcription loop-mediated isothermal amplification (RT-LAMP), a respective level of at least one biomarker associated with pressure injuries.

2. The method of claim 1, wherein the at least one biomarker is fatty acid binding protein-3 (FABP3) or fatty acid binding protein-4 (FABP4).

3. The method of claim 2, wherein detecting, in the biological sample, the respective level of the at least one biomarker associated with pressure injuries comprises detecting the respective levels of FABP3 and FABP4.

4. The method claim 1, wherein the biological sample comprises blood.

5. (canceled)

6. (canceled)

7. The method of claim 1, wherein the at least one biomarker is Kruppel-like factor 4, resistin, cyclin D1, sirtuin 2, dysferlin 2B, pyruvate, dehydrogenase kinase-4, dystrophin, or adiponectin.

8. The method of claim 1, wherein the biological sample comprises saliva or urine.

9. The method of claim 1, further comprising comparing the respective level of the at least one biomarker to a threshold, wherein the threshold is selected based on a level of the at least one biomarker relative to a level of a reference gene.

10. (canceled)

11. The method of claim 9, further comprising detecting the level of the reference gene.

12. The method of claim 11, wherein detecting the level of the reference gene comprises detecting the level of the reference gene using reverse transcription loop-mediated isothermal amplification (RT-LAMP) concurrently with detecting the respective level of the at least one biomarker associated with pressure injuries.

13. The method of claim 1, wherein detecting, in the biological sample, the respective level of the at least one biomarker associated with pressure injuries comprises using a microfluidics-based biochip to detect the respective level of the at least one biomarker associated with pressure injuries.

14. The method of claim 13, wherein using the microfluidics-based biochip to detect the respective level of the at least one biomarker associated with pressure injuries comprises detecting a colorimetric or fluorescence intensity output by the microfluidics-based biochip.

15. The method of claim 13, wherein using a microfluidics-based biochip to detect the respective level of the at least one biomarker associated with pressure injuries comprises using an apparatus comprising:
the microfluidics-based biochip, wherein the microfluidics-based biochip defines a channel, wherein the microfluidics-based biochip comprises at least one reagent within the channel that includes primers for performing RT-LAMP; and
at least one sensor that is configured to provide a signal indicative of the respective level of the at least one biomarker associated with pressure injuries.

16. The method of claim 15, wherein the at least one sensor comprises a colorimetric sensor, wherein a color detected by the colorimetric sensor corresponds to the respective level of the at least one biomarker associated with pressure injuries.

17. (canceled)

18. (canceled)

19. The method of claim 15, wherein the apparatus further comprises a UV source, wherein the at least one sensor comprises a fluorescence sensor, wherein an intensity of fluorescence detected by the fluorescence sensor corresponds to the respective level of the at least one biomarker associated with pressure injuries.

20. The method of claim 15, wherein the apparatus further comprises a computing device in communication with the at least one sensor, wherein the computing device provides an output in response to receiving a signal from the sensor that is above a threshold.

21. The method of claim 15, wherein the apparatus further comprises a computing device in communication with the at least one sensor, wherein the method comprises:
receiving, by the computing device, a first signal indicative of the respective level of the at least one biomarker associated with pressure injuries;
receiving, by the computing device, a second signal indicative of the respective level of a reference gene; and
providing, by the computing device, an output indicative of whether a ratio of the respective level of the at least one biomarker associated with pressure injuries to the respective level of a reference gene is above a relative threshold.

22. An apparatus comprising:
a microfluidics-based biochip, wherein the microfluidics-based biochip is configured to detect, in a biological sample, a respective level of at least one biomarker associated with pressure injuries:
a fluorescence or colorimetric sensor that is configured to provide a signal indicative of the respective level of the at least one biomarker associated with pressure injuries; and
a computing device in communication with the fluorescence or colorimetric sensor, wherein the computing device is configured to provide an output in response to receiving a signal from the fluorescence or colorimetric sensor that is indicative of the respective level of the at least one biomarker being above a threshold.

23. The apparatus of claim 22 wherein the at least one biomarker comprises FABP3 or FABP4.

24. The apparatus of claim 22, wherein the microfluidics-based biochip is configured to perform an RT-LAMP process to detect the respective level of the at least one biomarker.

25. The apparatus of claim 22, wherein the apparatus comprises an assembly comprising a first layer, a second layer, and an intermediate layer disposed between the first and second layers, wherein the intermediate layer defines a cutout that at least partly defines a channel within the assembly.

26. (canceled)

27. (canceled)

28. The apparatus of claim 22, wherein the computing device is configured to:
receive an input of a level of a reference gene present in the sample; and
set the threshold based on the level of the reference gene present.
